(19) United States
(12) Patent Application Publication
(10) Pub. No.: US 2008/0027125 A1
(43) Pub. Date: Jan. 31, 2008

(54) Fluvastatin sodium novel forms and preparation thereof

(76) Inventors: Tamas Koltai, Netanya (IL); Michael Pinchasov, Dover, NJ (US); Gustavo Frenkel, Beer Sheva (IL)

Correspondence Address: KENYON & KENYON LLP, ONE BROADWAY, NEW YORK, NY, 10004, US

(21) Appl. No.: 11/711,932

(22) Filed: Feb. 27, 2007

Related U.S. Application Data
(60) Provisional application No. 60/778,168, filed on Feb. 27, 2006.
(60) Provisional application No. 60/778,006, filed on Feb. 28, 2006.
(60) Provisional application No. 60/849,621, filed on Oct. 4, 2006.

(51) Int. Cl. C07D 209/04; A61K 31/403; A61P 3/00

(52) U.S. Cl. 514/419; 548/491

(57) ABSTRACT

Provided are a novel solid states of fluvastatin sodium and processes for preparation thereof.

### CROSS-REFERENCE TO RELATED APPLICATIONS

This application claims the benefit of U.S. Provisional Application Nos. 60/778,168, filed Feb. 27, 2006; 60/778,006, filed Feb. 28, 2006; and, 60/849,621, filed Oct. 4, 2006. The contents of which are incorporated herein by reference.

### FIELD OF THE INVENTION

The present invention relates to the antihypercholesterolemia and antilipidemia agent fluvastatin and, more particularly, to the solid state properties of its monosodium salt.

### BACKGROUND OF THE INVENTION

Complications of cardiovascular disease, such as myocardial infarction, stroke, and peripheral vascular disease account for half of the deaths in the United States. A high level of low density lipoprotein (LDL) in the bloodstream has been linked to the formation of coronary lesions which obstruct the flow of blood and can rupture and promote thrombosis. Goodman and Gilman, The Pharmacological Basis of Therapeutics 879 (9th ed. 1996). Reducing plasma LDL levels has been shown to reduce the risk of clinical events in patients with cardiovascular disease and in patients who are free of cardiovascular disease but who have hypercholesterolemia. Scandinavian Simvastatin Survival Study Group, 1994; Lipid Research Clinics Program, 1984a, 1984b.

Statin drugs are currently the most therapeutically effective drugs available for reducing the level of LDL in the blood stream of a patient at risk for cardiovascular disease. This class of drugs includes, inter alia, compactin, lovastatin, simvastatin, pravastatin and fluvastatin. The mechanism of action of statin drugs has been elucidated in some detail. They disrupt the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion of HMG-CoA to mevalonate, which is the rate determining step in the biosynthesis of cholesterol. Consequently, its inhibition leads to a reduction in the rate of formation of cholesterol in the liver.

[R*,S*-(E)]-(±)-7-[3-(4-fluorophenyl)-1-(1-methylethyl)-1H-indol-2-yl]-3,5-dihydroxy-6-heptenoic acid is a statin drug. It is known by the trivial name fluvastatin and has the molecular formula (I): 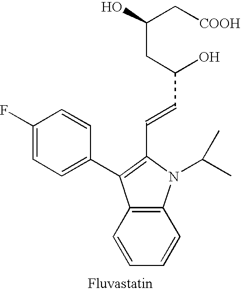
depicted in free acid form.

Fluvastatin is commercially available under the trade name Lescol®. Fluvastatin is supplied as a monosodium salt in capsules containing the equivalent of 20 and 40 mg of fluvastatin and in extended-release tablets containing the equivalent of 80 mg of fluvastatin. Fluvastatin and its sodium salt are described in U.S. Pat. No. 4,739,073 (“the '073 patent”). In Example 6(a) of the '073 patent, a methyl ester precursor of (±) fluvastatin is hydrolyzed with sodium hydroxide in methanol, which yielded, after evaporation of the methanol, crude fluvastatin sodium. In Example 6(b), the fluvastatin methyl ester is hydrolyzed with sodium hydroxide in ethanol. After evaporation of the ethanol, the residue is taken up in water and lyophilized. The lyophilized product is reported to have a melting point range of 194° C.-197° C. In Example 8, the sodium salt is prepared by ring opening of fluvastatin lactone with sodium hydroxide in ethanol as described in Example 6(b). The product of Example 8 is reported to produce an infrared spectrum in a KBr pellet with bands at: 3413, 2978, 2936, 1572 and 1216 cm⁻¹.

U.S. Pat. No. 6,124,340 (“the '340 patent”) describes fluvastatin Sodium Form B. According to the '340 patent, lyophilization of fluvastatin sodium that was performed in Examples 6(b) and 8 of the '073 patent yielded a mixture of fluvastatin sodium Form A and amorphous fluvastatin sodium. The '340 patent does not describe and characterize the amorphous form of Fluvastatin Sodium, it only mentions that Form A is less hygroscopic and has better light stability than the amorphous form.

Thus the '340 patent sets forth the spectroscopic properties of crystalline form B of fluvastatin sodium which is said to have low hygroscopicity and photostability. It is characterized by an infrared spectrum with bands at 3343, 2995, 1587, 1536, 1386, 1337, 1042 and 1014 cm⁻¹. Fluvastatin sodium Forms A and B are characterized by the following powder X-ray diffraction peak positions and intensities.

| | | |
|---|---|---|
| E22 | d (Δ) | I/I_{O}(%) |

Form A

| | | |
|---|---|---|
| 3.965 | 22.265 | 100 |
| 7.936 | 11.131 | 0.9 |
| 10.554 | 8.375 | 1.7 |
| 10.645 | 8.304 | 1.5 |
| 11.931 | 7.412 | 44.5 |
| 12.215 | 7.240 | 14.5 |
| 14.496 | 6.106 | 1.1 |
| 14.812 | 5.976 | 0.8 |
| 15.916 | 5.564 | 0.3 |
| 17.769 | 4.988 | 3.2 |
| 18.640 | 4.756 | 5.3 |
| 19.856 | 4.468 | 5.8 |
| 20.518 | 4.325 | 2.9 |
| 20.908 | 4.245 | 1.2 |
| 21.389 | 4.151 | 1.3 |
| 21.722 | 4.088 | 1.1 |
| 22.675 | 3.918 | 0.8 |
| 24.089 | 3.691 | 1.0 |
| 24.533 | 3.626 | 0.5 |
| 26.519 | 3.358 | 0.2 |
| 27.973 | 3.187 | 0.9 |
| 28.861 | 3.091 | |

Form B

| | | |
|---|---|---|
| 4.063 | 21.728 | 100 |
| 11.056 | 7.996 | 2.9 |
| 11.328 | 7.805 | 5.5 |
| 12.210 | 7.243 | 45.2 |
| 12.965 | 6.823 | 34.6 |
| 14.925 | 5.931 | 9.3 |
| 15.277 | 5.795 | 4.5 |
| 15.750 | 5.622 | 18.5 |
| 16.350 | 5.417 | 10.6 |
| 17.760 | 4.990 | 17.6 |
| 18.320 | 4.839 | 14.3 |
| 18.875 | 4.698 | 11.3 |
| 19.396 | 4.573 | 7.0 |
| 19.701 | 4.503 | 13.4 |
| 20.395 | 4.351 | 13.5 |
| 21.329 | 4.163 | 8.5 |
| 21.785 | 4.076 | 15.9 |
| 22.610 | 3.929 | 7.5 |
| 23.868 | 3.725 | 5.4 |
| 24.281 | 3.663 | 3.6 |
| 24.463 | 3.636 | 3.6 |
| 25.446 | 3.498 | 5.6 |
| 25.655 | 3.470 | 3.6 |
| 26.357 | 3.379 | 3.3 |
| 27.040 | 3.295 | 2.8 |
| 28.747 | 3.103 | 3.4 |
| 29.940 | 2.982 | 2.8 |
| 32.165 | 2.781 | 1.6 |
| 35.173 | 2.549 | 1.0 |
| 37.131 | 2.419 | 1.3 |

U.S. Patent Application Publication No. 2003/0032666 reports the existence of four crystal forms of fluvastatin monosodium called Forms C, D, E and F. The water content of the forms ranges between 3 and 32%. The new crystal forms of fluvastatin sodium were obtained by storing the samples of fluvastatin sodium in atmospheres having an humidity ranging between 20 and 90% relative humidity.

According to the '666 publication, the powder X-ray diffraction (PXRD) pattern of fluvastatin sodium Forms C, D, E and F possess characteristic peaks at the following d-values and qualitative intensities:

| | |
|---|---|
| d (Δ) | Intensity |

Form C

| | |
|---|---|
| 23.8 | (vs) |
| 11.8 | (w) |
| 7.8 | (vs) |
| 7.6 | (vw) |
| 7.4 | (vw) |
| 6.4 | (vw) |
| 6.1 | (vw) |
| 5.90 | (w) |
| 5.00 | (vw) |
| 4.88 | (w) |
| 4.73 | (m) |
| 4.56 | (w) |
| 4.40 | (vw) |
| 4.12 | (vw) |
| 4.03 | (vw) |
| 3.96 | (vw) |
| 3.50 | (vw) |
| 3.36 | (vw) |
| 2.93 | (vw) |

Form D

| | |
|---|---|
| 24.6 | (vs) |
| 12.5 | (w) |
| 8.3 | (vs) |
| 7.4 | (vw) |
| 6.2 | (m) |
| 4.97 | (w) |
| 4.85 | (vw) |
| 4.52 | (vw) |
| 4.40 | (vw) |
| 4.14 | (vw) |
| 3.96 | (vw) |
| 3.41 | (vw) |
| 3.10 | (vw) |

Form E

| | |
|---|---|
| 27.6 | (m) |
| 13.9 | (vw) |
| 9.2 | (m) |
| 8.5 | (vw) |
| 8.1 | (vw) |
| 7.4 | (vw) |
| 6.9 | (s) |
| 6.1 | (vw) |
| 4.98 | (m) |
| 4.77 | (m) |
| 4.63 | (m) |
| 4.15 | (w) |
| 4.03 | (w) |
| 3.97 | (vw) |
| 3.52 | (vw) |
| 3.33 | (vw) |
| 3.08 | (vw) |
| 2.99 | (vw) |

Form F

| | |
|---|---|
| 29.6 | (w) |
| 14.8 | (vw) |
| 9.9 | (w) |
| 8.6 | (vw) |
| 8.3 | (vw) |
| 7.4 | (s) |
| 6.6 | (vw) |
| 6.2 | (vw) |
| 5.93 | (w) |
| 5.03 | (m) |
| 4.94 | (m) |
| 4.35 | (vw) |
| 4.23 | (w) |
| 3.98 | (vw) |
| 3.54 | (vw) |
| 2.98 | (vw) |

wherein
(vs) = very strong intensity;
(s) = strong intensity;
(m) = medium intensity;
(w) = weak intensity;
and (vw) = very weak intensity.

It also deserves mention that International Publication No. WO 02/36563 describes crystal forms of enantiomerically pure [3R,5S] and [3S,5R] fluvastatin sodium.

Additional crystalline forms of fluvastatin sodium (and processes for their preparation) are also mentioned in the following PCT publications: WO04/96765, WO04/113291 and WO04/113292, WO05/003286, WO05/080332. WO06/21967, WO06/30304, WO06/085338, WO06/085338 and WO06/038219.

Anhydrous amorphous Fluvastatin Sodium is described in US Patent Application Publication No. 2005/0209259, and is characterized by an XRD diffractogram.

The present invention relates to fluvastatin sodium and the properties that it exhibits in its solid phase. The occurrence of different crystal forms (polymorphism) is a property of some molecules and molecular complexes. A single molecule, like the fluvastatin in formula (I) or a salt complex like fluvastatin sodium, may give rise to a variety of solids having distinct physical properties like melting point, X-ray diffraction pattern, infrared absorption fingerprint and NMR spectrum. The crystalline form may give rise to thermal behavior different from that of the amorphous material or another crystalline form. Thermal behavior is measured in the laboratory by such techniques as capillary melting point, thermogravimetric analysis (“TGA”) and differential scanning calorimetry (“DSC”) and can be used to distinguish some polymorphic forms from others. The differences in the physical properties of different crystalline forms result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. These properties can be influenced by controlling the conditions under which the salt is obtained in solid form.

Exemplary solid state physical properties include the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

One of the most important physical properties of polymorphs of active pharmaceutical ingredients is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. On the other hand, this is not advantageous where the effectiveness of a drug correlates with peak bloodstream levels of the drug, as in the case of statin drugs. With a statin drug, provided the drug is rapidly absorbed by the GI system, a more rapidly dissolving form is likely to exhibit increased effectiveness over a comparable amount of a more slowly dissolving form.

It is often the case that the most rapidly dissolving solid state of a compound is amorphous.

The discovery of yet other forms of fluvastatin sodium is desirable. The discovery of new forms of a pharmaceutically useful compound provides a new opportunity to improve the performance characteristics of a pharmaceutical product by enlarging the repertoire of materials that a formulation scientist has available for designing a formulation. For example, new crystalline forms can be used to design a pharmaceutical dosage form of a drug, a targeted release profile, consistent dosing, or other desired characteristic. New forms of fluvastatin sodium have now been discovered.

### SUMMARY OF THE INVENTION

In one embodiment the present invention provides a crystalline form of fluvastatin sodium characterized by a PXRD pattern with a strong peak at about 3.7±0.2 degrees two-theta.

In another embodiment the present invention provides a process for preparing the above crystalline form comprising spray drying a solution of fluvastatin sodium in methanol at an outlet temperature of at least about 90° C.

In another embodiment the present invention provides amorphous fluvastatin sodium having at least about 3% water by weight.

In another embodiment the present invention provides a process for preparing amorphous fluvastatin sodium comprising spray drying a solution of fluvastatin sodium in methanol, wherein the gas is at an outlet temperature of less than about 90° C.

In another embodiment the present invention provides a process for preparing amorphous fluvastatin sodium comprising spray drying a solution of fluvastatin sodium in acetone.

In another embodiment the present invention provides a crystalline form of fluvastatin sodium characterized by X-ray powder diffraction reflections at about 3.7, 9.3, 10.0 and 11.8±0.2 degrees two-theta.

In another embodiment the present invention provides a process for preparing the above crystal form comprising precipitating fluvastatin sodium from a mixture of fluvastatin sodium in acetonitrile; milling the wet fluvastatin sodium and drying the fluvastatin sodium, to obtain the fluvastatin sodium form.

In another embodiment the present invention provides a pharmaceutical composition comprising at least one of fluvastatin sodium crystalline forms described above and/or amorphous fluvastatin sodium having at least about 3% water and mixtures thereof and a pharmaceutically acceptable excipient.

### BRIEF DESCRIPTION OF THE FIGURES

FIG. 1 depicts an XRD diffractogram of amorphous Fluvastatin Sodium obtained from Example 1/a.

FIG. 1a presents the calculation of the peak width at half height for the amorphous Fluvastatin Sodium obtained from Example 1/a.

FIG. 2 presents the XRD diffractogram of Fluvastatin Sodium Form CVI obtained from Example 1/b.

FIG. 2a presents the calculation of the peak width at half height for Fluvastatin Sodium Form CVI obtained from Example 1/b.

FIG. 3 presents the XRD diffractogram of amorphous Fluvastatin Sodium obtained from Example 2.

FIG. 4 presents the XRD diffractogram of Fluvastatin Sodium Form CVII.

### DETAILED DESCRIPTION OF THE PREFERRED EMBODIMENTS

As used herein, the term “polymorphically stable”, in reference to fluvastatin sodium crystalline form, relates to a crystalline fluvastatin sodium polymorph that does not convert into another form of fluvastatin sodium by more than 10% by weight when:

a) exposed to relative humidity of about 0% to about 60%, for about 10 days; and/or

b) heated to a temperature of about 50° C.-100° C. for about 1 to 3 hours.

As used herein, the term “polymorphically stable”, in reference to amorphous fluvastatin sodium crystalline form, relates to amorphous fluvastatin sodium polymorph that does not convert into another form of fluvastatin sodium by more than 10% by weight when exposed to relative humidity of about 0% to about 60%, for about 10 days.

As used herein, the term “strong peak” refers to a sharp peak (in contrast to a broad peak) which its width at half height is of about 0.2-0.4 degrees two-theta.

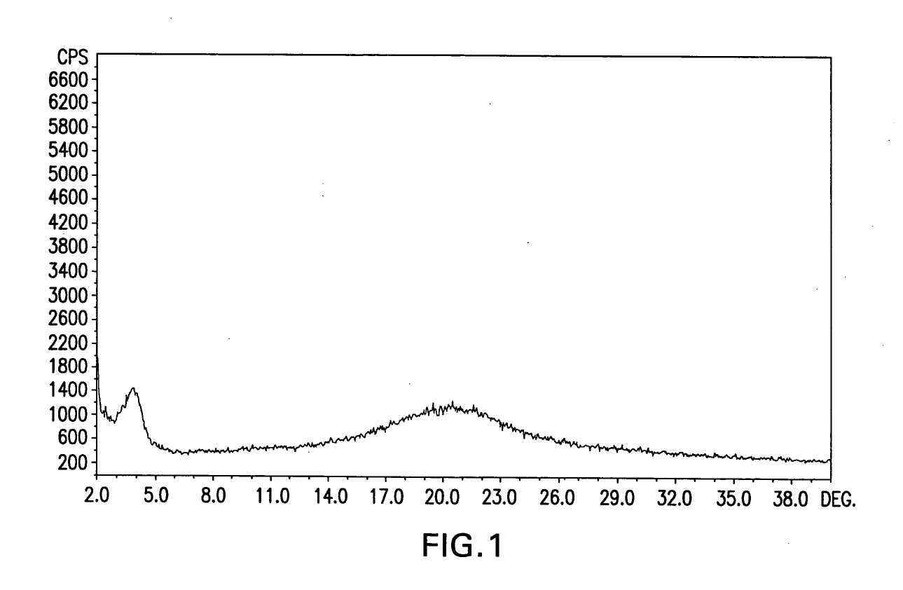

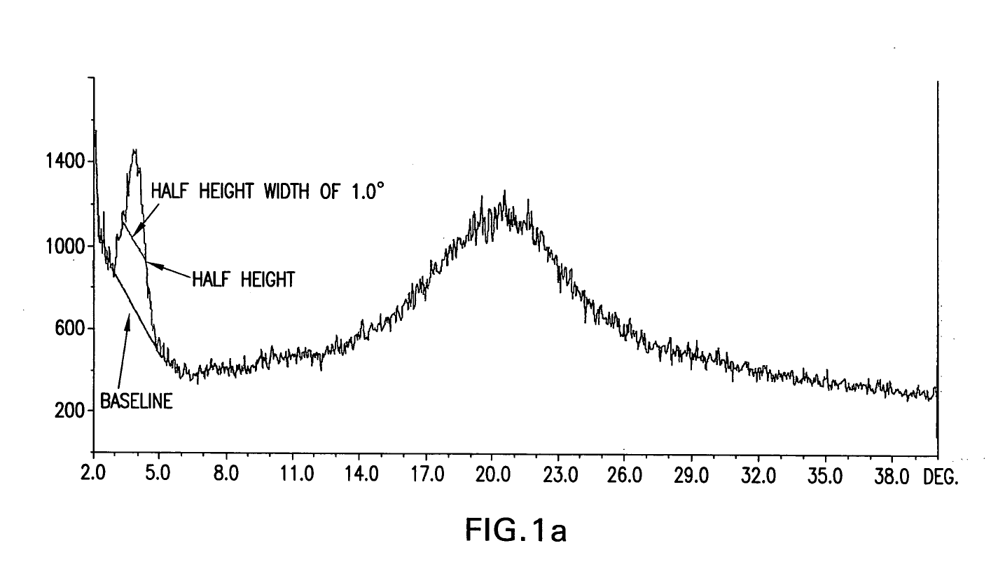

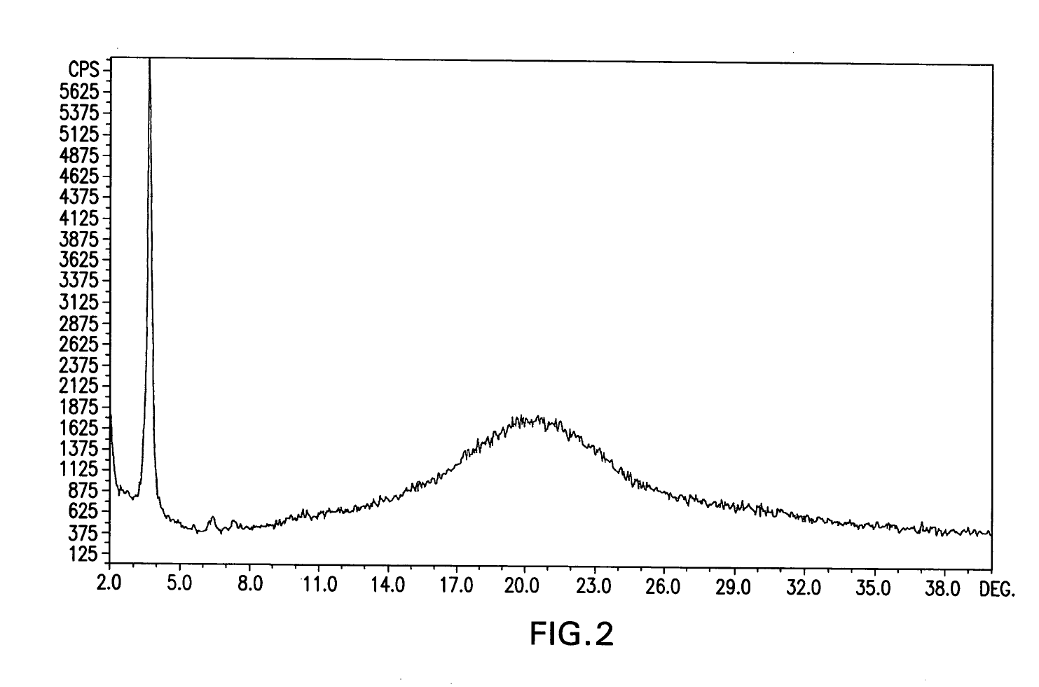

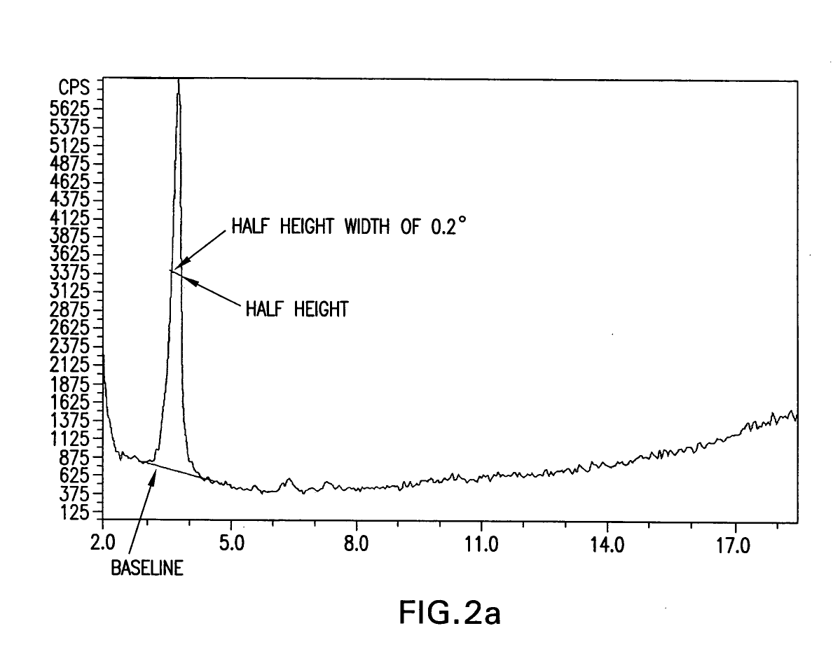

The present invention provides a crystalline form of fluvastatin sodium characterized by a PXRD pattern with a strong peak at about 3.7±0.2 degrees two-theta (denominated Form CVI). Form CVI can also be characterized by X-ray powder diffraction reflections at about 3.7, 6.4 and 7.3±0.2, substantially as depicted in FIG. 2. The XRD diffractogram of Form CVI demonstrates a strong peak at 3.7±0.2 degrees two-theta, that is a peak which its width at half height is of about 0.2-0.4 degrees two-theta, while the width at half height of the peak at 3.9±0.2 degrees two-theta of the amorphous form is of about 1.0 degrees two-theta or higher. The peak width calculations are presented on FIGS. 1a and 2a.

Form CVI is polymorphically stable.

Specifically, fluvastatin Sodium Form CVI is stable upon exposure to relative humidities between about 0% to about 60%. When maintained at relative humidities between about 0% to about 60% for 10 days, Form CVI remains stable. Form CVI contains at least about 3% water. More specifically, fluvastatin sodium Form CVI does not convert into fluvastatin sodium Form LXVI by more than 10%. Fluvastatin sodium Form CVI is also stable upon heating to about 80° C. for about 1 hour. After maintaining Form CVI under these conditions, it contains about 2% water. More specifically, fluvastatin sodium Form CVI does not convert into fluvastatin sodium Form LXVI by more than 10%.

Fluvastatin Sodium form LXVI is described in US 2005/0032884, and is characterized by a PXRD having peaks at about 3.6, 10.8, 17.8, 18.3 and 21.6±0.2 degrees two-theta.

The present invention provides a process for preparing Form CVI, comprising spray drying a solution of fluvastatin sodium in methanol wherein the gas is at an outlet temperature of at least about 90° C. More preferably, the gas is at an outlet temperature of about 110° C. to about 120° C.

Preferably, the solution is spray-dried at an inlet temperature of about 170° C. to about 220° C., more preferably about 200° C.

The term “spray drying” broadly refers to processes involving breaking up liquid mixtures into small droplets (atomization) and rapidly removing solvent from the mixture. In a typical spray drying apparatus, a strong driving force evaporates the solvent from the droplets, which may be provided by providing a drying gas. Spray drying processes and equipment are described in Perry's Chemical Engineer's Handbook, pgs. 20-54 to 20-57 (Sixth Edition 1984).

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream from the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. The process of the invention is not limited to the use of such drying apparatuses as described above.

Spray drying may be performed in a conventional manner in the processes of the present invention (see, e.g., Remington: The Science and Practice of Pharmacy, 19th Ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The fluvastatin sodium product produced by spray drying may be recovered by techniques commonly used in the art, such as using a cyclone or a filter.

The drying gas used in the process of the present invention may be any suitable gas, although inert gases such as nitrogen, nitrogen-enriched air, and argon are preferred.

Inlet or outlet temperatures may be varied, if necessary, depending on the equipment, gas, or other experimental parameters. For example, it is known that the outlet temperature may depend on parameters such as aspirator rate, air humidity, inlet temperature, spray air flow, feed rate or concentration.

The present invention also provides amorphous fluvastatin sodium, containing at least about 3% water by weight. Preferably, the amorphous fluvastatin sodium contains about 3% to about 5.2% water by weight.

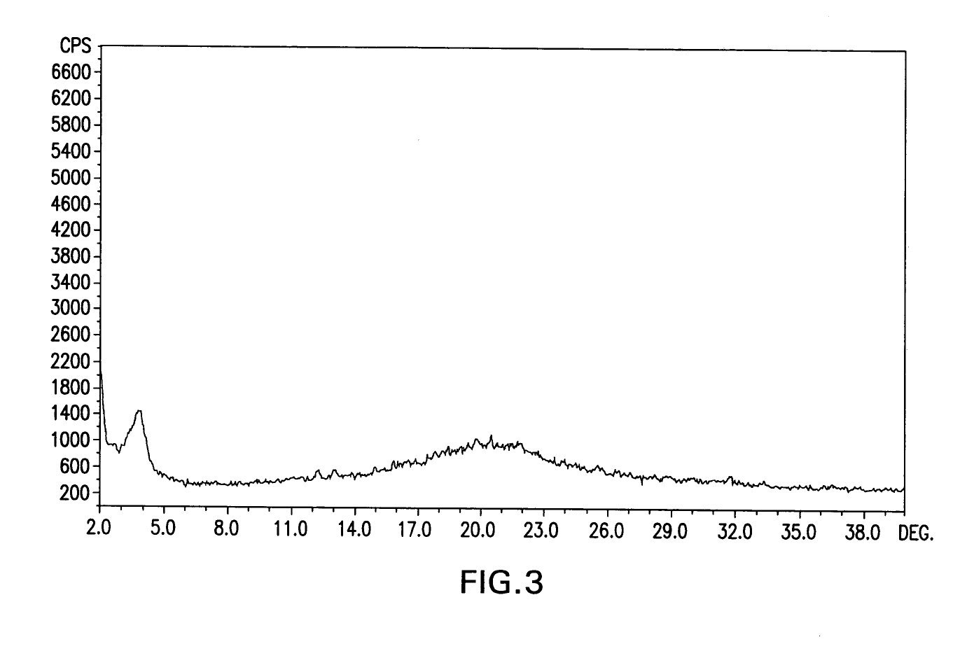

The amorphous fluvastatin sodium of the present invention exhibits different dissolution characteristics and therefore is apt to have good bioavailability qualities. This amorphous form is characterized by a broad PXRD pattern, as substantially depicted in FIGS. 1 and 3.

The present invention provides a process for preparing amorphous fluvastatin sodium comprising spray drying a solution of fluvastatin sodium in methanol wherein the gas is at an outlet temperature of less than about 90° C. Preferably, the gas is at an outlet temperature of about 30° C. to about 38° C., most preferably, an outlet temperature of about 34° C.

Preferably, the solution is spray-dried at an inlet temperature of about room temperature to about 170° C., more preferably, about 50° C.

Preferably, the obtained amorphous fluvastatin sodium contains at least about 3% water by weight.

The present invention also provides a process for preparing amorphous fluvastatin sodium comprising spray drying a solution of fluvastatin sodium in acetone.

Preferably, the solution is spray-dried at an inlet temperature of about 95° C. to about 105° C., more preferably about 100° C. Preferably, the outlet temperature is about room temperature to about 90° C., more preferably about 55° C. to about 65° C., most preferably about 60° C. to about 63° C. Preferably, the solution is heated to a temperature of about room temperature to reflux prior to spray drying. Most preferably, the solution is heated to a temperature of about 50° C.

Preferably, the obtained amorphous fluvastatin sodium contains at least about 3% water by weight.

The above amorphous fluvastatin sodium is polymorphically stable.

Specifically, the amorphous Fluvastatin Sodium, having at least about 3% water is stable upon exposure to relative humidities between 0% and 60%. This amorphous fluvastatin sodium was found stable when exposed to these relative humidities for 10 days and contained at least 3% water. More specifically, amorphous fluvastatin sodium does not convert into fluvastatin sodium Form LXVI by more than 10%.

The amorphous form of fluvastatin sodium of the present invention contains no more than about 10% more preferably no more than about 5% of crystalline fluvastatin sodium. This determination may be made by calculating area under XRD peaks.

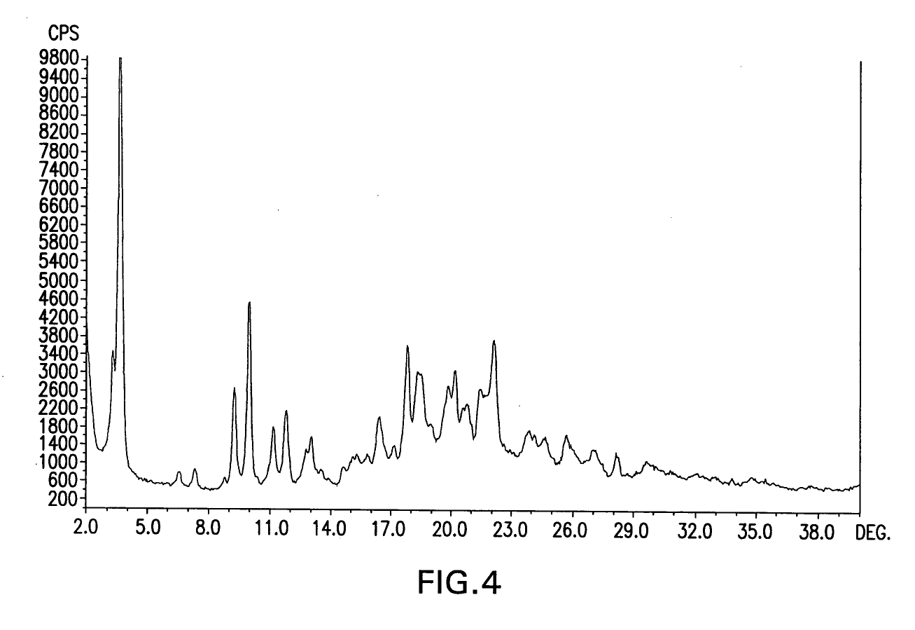

In another aspect, the present invention provides a crystalline form of fluvastatin sodium characterized by X-ray powder diffraction reflections at about 3.7, 9.3, 10.0 and 11.8±0.2 degrees two-theta (denominated Form CVII). Form CVII may be further characterized by a X-ray powder diffraction pattern with peaks at about 3.4, 6.6, 7.4, 16.4, and 20.1±0.2 degrees two-theta. A typical powder x-ray diffractogram for Form CVII is substantially depicted in FIG. 4.

The present invention provides a process for preparing Form CVII comprising: precipitating fluvastatin sodium from a mixture of fluvastatin, sodium hydroxide and acetonitrile; milling the wet fluvastatin sodium and drying the fluvastatin sodium, to obtain fluvastatin sodium Form CVII.

In one embodiment, a sodium base is combined with an ester of fluvastatin in acetonitrile. The ester is preferably a C₁-C₄ alkyl ester such as a methyl or t-butyl ester. The base may first be dissolved in water, to which the ester and acetonitrile are combined with. Examples of bases include sodium hydroxide. The reaction mixture can be heated to accelerate the hydrolysis. Heating may be carried out from about room temperature to about reflux temperature of the solvent. The reaction mixture may also be stirred for a suitable time. The pH of the reaction mixture is preferably kept below about 10, more preferably about 8 to about 10. An additional amount of acetonitrile may be added to precipitate the fluvastatin sodium. The precipitation can occur at about room temperature. The precipitate may then be recovered by conventional techniques such as filtration, washed with water and/or an organic solvent.

The fluvastatin sodium precipitates in aggregates or lumps. These aggregates can be broken down by milling.

The milled material is then dried under ambient or reduced pressure, and/or elevated temperature. It also may be stirred during the drying process. A suitable drying condition is about 30° C. to about 60° C., more preferably about 40° C. to about 50° C. A suitable pressure is ambient or below room pressure, preferably below about 100 mmHg. In one embodiment, the precipitate is dried at about 40° C. at a pressure of less than about 100 mmHg.

As one skilled in the art will appreciate, the time required to obtain this fluvastatin sodium form will vary depending upon, among other factors, the amount of wet fluvastatin sodium to be dried and the drying temperature, and can be determined by taking periodic XRD's

Preferably, the amorphous fluvastatin sodium, as well as the crystalline forms described above, have a maximum particle size of less than about 500 μm, more preferable less than 300 μm, even more preferable less than 200 μm, yet even more preferable less than 100 μm and most preferable less than 50 μm.

For measuring particle size the following main methods may be employed: sieves, sedimentation, electrozone sensing (coulter counter), microscopy, Low Angle Laser Light Scattering (LALLS).

The present invention further provides a pharmaceutical composition comprising at least one of the fluvastatin sodium crystalline forms described above and/or amorphous fluvastatin sodium having at least about 3% water and a pharmaceutically acceptable excipient.

Fluvastatin exerts an antihypercholesterolemia and antihyperlipidemia effect in mammals, especially humans. Accordingly, fluvastatin sodium Forms CVI and CVII and/or amorphous fluvastatin sodium are useful for delivering fluvastatin to the gastrointestinal tract, bloodstream and liver of humans and other mammals suffering from or at risk of atherosclerosis. In particular, it is useful as active ingredients in pharmaceutical compositions and dosage forms. For this purpose, they may be formulated into a variety of compositions and dosage forms for administration to humans and animals.

Pharmaceutical compositions of the present invention contain fluvastatin sodium Forms CVI and CVII and/or amorphous fluvastatin sodium or mixtures thereof with other crystalline forms of fluvastatin sodium, optionally in mixtures with one or more other active ingredient(s). In addition to the active ingredient(s), the pharmaceutical compositions of the present invention may contain one or more excipients. Excipients are added to the composition for a variety of purposes.

Diluents increase the bulk of a solid pharmaceutical composition and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flow properties of non-compacted solid composition and improve the accuracy of dosing. Excipients that may function as glidants include colloidal silicon dioxide, magnesium trisilicate, powdered cellulose, starch, talc and tribasic calcium phosphate.

When a dosage form such as a tablet is made by compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, at least one of the fluvastatin sodium crystalline forms described above and/or amorphous fluvastatin sodium having at least about 3% water and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

A liquid composition according to the present invention may also contain a buffer such as guconic acid, lactic acid, citric acid or acetic acid, sodium guconate, sodium lactate, sodium citrate or sodium acetate.

Selection of excipients and the amounts to use may be readily determined by the formulation scientist based upon experience and consideration of standard procedures and reference works in the field.

The solid compositions of the present invention include powders, granulates, aggregates and compacted compositions. The dosage forms include dosage forms suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges as well as liquid syrups, suspensions and elixirs.

An especially preferred dosage form of the present invention is a capsule containing the composition, preferably a powdered or granulated solid composition of the invention, within either a hard or soft shell. The shell may be made from gelatin and optionally contain a plasticizer such as glycerin and sorbitol, and an opacifying agent or colorant. An especially preferred capsule filling contains, in addition to one or more of the fluvastatin sodium crystalline forms of this invention, the excipients magnesium stearate, microcrystalline cellulose, pregelatinized starch, sodium lauryl sulfate and talc.

Another especially preferred dosage form of this invention is a compressed tablet that contains, in addition to one or more of the fluvastatin sodium crystalline forms of this invention, the excipients microcrystalline cellulose, hydroxypropyl cellulose, hydroxypropylmethyl cellulose, potassium bicarbonate, povidone, magnesium stearate, iron oxide yellow, titanium dioxide, and polyethylene glycol 8000.

The active ingredient and excipients may be formulated into compositions and dosage forms according to methods known in the art.

A composition for tableting or capsule filing may be prepared by wet granulation. In wet granulation some or all of the active ingredients and excipients in powder form are blended and then further mixed in the presence of a liquid, typically water, that causes the powders to clump up into granules. The granulate is screened and/or milled, dried and then screened and/or milled to the desired particle size. The granulate may then be tableted or other excipients may be added prior to tableting such as a glidant and or lubricant.

A tableting composition may be prepared conventionally by dry blending. For instance, the blended composition of the actives and excipients may be compacted into a slug or a sheet and then comminuted into compacted granules. The compacted granules may be compressed subsequently into a tablet.

As an alternative to dry granulation, a blended composition may be compressed directly into a compacted dosage form using direct compression techniques. Direct compression produces a more uniform tablet without granules. Excipients that are particularly well suited to direct compression tableting include microcrystalline cellulose, spray dried lactose, dicalcium phosphate dihydrate and colloidal silica. The proper use of these and other excipients in direct compression tableting is known to those in the art with experience and skill in the particular formulation challenges of direct compression tableting.

A capsule filling of the present invention may comprise any of the aforementioned blends and granulates that were described with reference to tableting, only they are not subjected to a final tableting step.

Capsules, tablets and lozenges and other unit dosage forms preferably contain a dosage equivalent to from about 10 to about 100 mg fluvastatin. Preferably the dosage is equivalent to from about 20 to about 80 mg of fluvastatin. More particularly, immediate or uncontrolled release dosage forms preferably contain the equivalent of from about 20 to about 40 mg of fluvastatin and extended release dosage forms preferably contain the equivalent of from about 60 to about 100 mg of fluvastatin, more preferably about 80 mg of fluvastatin.

Having thus described the present invention with reference to certain preferred embodiments, the processes for producing Fluvastatin sodium Forms CVI and CVII and/or amorphous fluvastatin sodium of the present invention and techniques suitable for identifying them are further illustrated by the examples which follow. These examples are provided for illustrative purposes only and are not intended to limit the invention in any way.

### EXAMPLES

### General

In the following examples, the forms of Fluvastatin Sodium were identified using a Scintag X-ray powder diffractometer model X'TRA with a solid state detector. Copper radiation of 1.5418 Å was used. The sample holder was a round standard aluminum sample holder with rough zero background. The scanning parameters were range: 2-40 degrees two-theta; scan mode: continuous scan; step size: 0.05 deg.; and a rate of 5 deg/min.

To determine the weight loss by thermal gravimetric analysis (TGA), the sample was heated from about 25° C. to about 200° C. at a heating rate of about 10° C. per minute, while purging with nitrogen gas at a flow rate of 40 ml/min.

The water content of the samples was determined by the method known as Karl Fisher (KF).

### Example 1

### Preparation of Amorphous Fluvastatin Sodium and Form CVI

Fluvastatin (5 g) was dissolved in methanol (20 ml) at room temperature. The solution was divided into two parts. The both parts were spray dried using a Buchi mini spray dryer B-290 using a standard nozzle 0.7 mm in diameter with a nozzle cap of 1.4 mm.

### Example 1/a

### Preparation of Amorphous Fluvastatin Sodium

At the first part the nitrogen gas was at an inlet temperature of 50° C. The evaporated solvent and nitrogen left the spray dryer at a temperature of 34-38° C. The obtained sample was analyzed by XRD and found to be amorphous Fluvastatin Sodium (see FIG. 1)

Water content by KF was 5.1%.

### Example 1/b

### Preparation of Fluvastatin Sodium Form CV1

At the second part the nitrogen gas was at an inlet temperature of 200° C. The evaporated solvent and nitrogen left the spray dryer at a temperature of 112-115° C. The obtained sample was analyzed by XRD and found to be Fluvastatin Sodium Form CVI (see FIG. 2). Weight loss of the sample was 4.5%, water content by KF was 4.5%.

### Example 2

### Preparation of Amorphous Fluvastatin Sodium

Fluvastatin (2 g) was added into Acetone (50 ml). The mixture was heated to 50° C. and spray dried using a Buchi mini spray dryer B-290 using a standard nozzle 0.7 mm in diameter with a nozzle cap of 1.4 mm. Nitrogen gas was at an inlet temperature of 100° C. The evaporated solvent and nitrogen left the spray dryer at a temperature of 60-63° C. The obtained sample was analyzed by XRD and found to be amorphous Fluvastatin Sodium (see FIG. 3). Water content by KF was 5.2%.

### Example 3

### Heating of Fluvastatin Sodium Form CVI

Fluvastatin Na Form CVI (0.5 G) Obtained from Example 1/B was Placed to Traditional Oven on 80 C during 1 hour. The obtained sample was analyzed by KF and XRD and found to be Fluvastatin Sodium Form CVI with 2% Water Content.

### Example 4

### Storing Amorphous Fluvastatin Sodium at Room Temperature Under 0-60% RH Conditions

Two amorphous Fluvastatin Sodium samples (each of about 0.25 g) obtained from example 2 were stored under 0% and under 60% RH during 10 days. Before and after the storage time the sample was analyzed by XRD. The sample stored under 0% and under 60% RH found to be amorphous Fluvastatin Sodium by XRD at the end of the storage time. The sample stored under 0% RH had 4% water content.

### Example 5

### Storing Fluvastatin Sodium Form CVI at Room Temperature Under 0-60% RH Conditions

Two fluvastatin Sodium Form CVI samples (each of about 0.25 g) obtained from example 1/b were stored under 0% and under 60% RH during 10 days. Before and after the storage time the sample was analyzed by XRD and KF. The sample stored under 0% and under 60% RH found to be Fluvastatin Sodium Form CVI by XRD at the end of the storage time. The sample stored under 0% RH had 3% water content.

### Example 6

### Storing Fluvastatin Sodium Form CVI and Amorphous at Room Temperature Under 80-100% RH Conditions

Amorphous Fluvastatin Sodium (0.5 g) obtained from example 2 and Fluvastatin Sodium Form CVI obtained from example 1/b were stored under 100% RH during 10 days and found to be Fluvastatin Na Form LXVI by XRD. The samples stored under 100% RH had about 25% water content.

### Example 7

### Preparation of Fluvastatin Sodium Form CVII

4 kg of NaOH pearls and 68 kg of water are charged into a reactor and stirred until a clear solution is obtained. Then, 45 kg of FDE-tBu are charged into the reactor followed by 144 kg of Acetonitrile.

The mixture is heated to 35±3° C. and stirred for 2-4 hours at the same temperature.

pH is checked to be in range of 8.5-10. In case of pH>10, it is corrected with HCl 32% solution until pH is between 8.5-10 is obtained.

The mixture is then filtrated to remove foreign matters and brought to 25±3° C.

576 kg of Acetonitrile are dropped keeping the temperature 25±° C. Precipitation occurs during the addition.

The mixture is stirred at 25±3° C. for at least 3 hours.

The mixture is filtered and the material cake is washed with a mixture of 10 kg of water and 28 kg of Acetonitrile, previously prepared.

31.10 kg of wet FLV Na, were milled to break lumps. The milled material was charged into a vacuum oven and dried at temperature of 40±3° C. and vacuum ≦100 mmHg, following the detailed drying pattern:

1. First 2 hours without stirring.

2. Stirring for 1 minute at 1-3 rpm.

3. 2.5 hours stirring every 0.5-1.5 hours at 1-3 rpm for 1-3 minutes.

4. Keep drying with continuous stirring until dryness.

11.15 kg of dry material were obtained, analyzed by XRD and shown to be Fluvastatin Sodium Form CVII.

Having thus described the invention with reference to particular preferred embodiments and illustrated it with examples, those in the art may appreciate modifications to the invention as described and illustrated that do not depart from the spirit and scope of the invention as defined by the claims which follow.

What is claimed is:

1. A crystalline form of fluvastatin sodium characterized by a PXRD pattern with a strong peak at about 3.7±0.2 degrees two-theta.

2. The crystal form of claim 1, wherein the crystalline form is further characterized by a PXRD pattern with peaks at about 3.7, 6.4 and 7.3±0.2.

3. The crystalline form of claim 1, wherein the crystalline form is further characterized by a PXRD pattern as substantially depicted in FIG. 2.

4. The crystalline form of claim 1, wherein the crystalline form is polymorphically stable.

5. The crystalline form of claim 1, wherein the crystalline is stable upon exposure to relative humidities of about 0% to about 60% for 10 days.

6. The crystalline form of claim 5, wherein the crystalline form contains at least about 3% water by weight.

7. The crystalline form of claim 1, wherein the crystalline form is stable upon heating to about 80° C. for about 1 hour.

8. The crystalline form of claim 7, wherein the crystalline form contains about 2% water by weight.

9. The crystalline form of claim 1, wherein the crystalline form does not convert into fluvastatin sodium characterized by a PXRD having peaks at about 3.6, 10.8, 17.8, 18.3 and 21.6±0.2 degrees two-theta by more than 10% by weight.

10. A process for preparing the crystalline form of claim 1, comprising spray drying a solution of fluvastatin sodium in methanol at an outlet temperature of at least about 90° C.

11. The process of claim 10 wherein the outlet temperature is between about 110° C. to about 120° C.

12. The process of claim 10 wherein the solution is spray-dried at an inlet temperature of about 170° C. to about 220° C.

13. The process of claim 10 wherein the inlet temperature is about 200° C.

14. A process for preparing amorphous fluvastatin sodium comprising spray drying a solution of fluvastatin sodium in methanol, wherein the gas is at an outlet temperature of less than about 90° C.

15. The process of claim 14, wherein the amorphous fluvastatin sodium contains at least about 3% water by weight.

16. The process of claim 14, wherein the gas is at an outlet temperature of about 30° C. to about 38° C.,

17. The process of claim 14, wherein the gas is at an outlet temperature of about 34° C.

18. The process of claim 14, wherein the solution is spray-dried at an inlet temperature of about room temperature to about 170° C.

19. The process of claim 18 wherein the inlet temperature is about 50° C.

20. A process for preparing amorphous fluvastatin sodium comprising spray drying a solution of fluvastatin sodium in acetone.

21. The process of claim 20, wherein the amorphous fluvastatin sodium contains at least about 3% water by weight.

22. The process of claim 20 wherein the solution is spray-dried at an inlet temperature of about 95° C. to about 105° C.

23. The process of claim 22, wherein the inlet temperature is about 100° C.

24. The process of claim 20, wherein the outlet temperature is about room temperature to about 90° C.

25. The process of claim 24, wherein the outlet temperature is about 55° C. to about 65° C.

26. The process of claim 25, wherein the outlet temperature is about 60° C. to about 63° C.

27. The process of claim 20 wherein the solution is heated to a temperature of about room temperature to reflux prior to spray drying.

28. The process of claim 27 wherein the solution is heated to a temperature of about 50° C.

29. A crystalline form of fluvastatin sodium characterized by X-ray powder diffraction reflections at about 3.7, 9.3, 10.0 and 11.8±0.2 degrees two-theta.

30. The crystal form of claim 29 wherein the crystal form is further characterized by a X-ray powder diffraction pattern with peaks at about 3.4, 6.6, 7.4, 16.4, and 20.1±0.2 degrees two-theta.

31. The crystal form of claim 29, wherein the crystal form has a typical powder x-ray diffractogram form as substantially depicted in FIG. 4.

32. A process for preparing crystal form of claim 29 comprising precipitating fluvastatin sodium from a mixture of fluvastatin sodium in acetonitrile; milling the wet fluvastatin sodium and drying the fluvastatin sodium, to obtain the fluvastatin sodium form.

33. The process of claim 32, wherein a sodium base is combined with an ester of fluvastatin in acetonitrile, thereby hydrolyzing the ester and forming fluvastatin sodium in acetonitrile.

34. The process of claim 33, wherein the ester is preferably a C₁-C₄ alkyl ester.

35. The process of claim 34, wherein the ester is a methyl or t-butyl ester.

36. The process of claim 33, wherein the base first is first dissolved in water, to which the ester and a acetonitrile are added.

37. The process of claim 36, wherein the base is sodium hydroxide.

38. The process of claim 33 wherein the reaction mixture is heated to accelerate the hydrolysis.

39. The process of claim 38, wherein heating is carried out from about room temperature to about reflux temperature of the solvent.

40. The process of claim 32, wherein pH of the reaction mixture is kept below about 10.

41. The process of claim 40, wherein pH of the reaction mixture is about 8 to about 10.

42. The process of claim 32, wherein an additional amount of acetonitrile is added to precipitate the fluvastatin sodium.

43. The process of claim 32, wherein the precipitation is carried out at about room temperature.

44. The process of claim 42, further comprising recovering the precipitate.

45. The process of claim 32, wherein the milled material is dried.

46. The process of claim 35, wherein drying is carried out at a temperature of about 30° C. to about 60° C.

47. The process of claim 46, wherein the temperature is about 40° C. to about 50° C.

48. The process of claim 45, wherein drying is carried out at a pressure of below about 100 mmHg.

49. A pharmaceutical composition comprising at least one of fluvastatin sodium forms according to the preceding claims and at least one pharmaceutically acceptable carrier.

50. A process for preparing a pharmaceutical composition comprising combining (i) at least one form of fluvastatin sodium selected from the group consisting of a crystalline form of fluvastatin sodium characterized by a PXRD pattern with a strong peak at about 3.7±0.2 degrees two-theta, a crystalline form of fluvastatin sodium characterized by X-ray powder diffraction reflections at about 3.7, 9.3, 10.0 and 11.8±0.2 degrees two-theta and mixtures thereof and (ii) a pharmaceutically acceptable excipient.

51. A method of reducing cholesterol levels in a mammal comprising administering the pharmaceutical composition of claim 49 to the mammal.

52. The fluvastatin sodium prepared by the process of claim 10.

53. The fluvastatin sodium prepared by the process of claim 32.
